(19) United States
(12) Patent Application Publication
(10) Pub. No.: US 2026/0091167 A1
(43) Pub. Date: Apr. 2, 2026

(54) ELECTRIC NASAL SUCTION DEVICE WITH SECONDARY AIR GAP CHAMBER

(71) Applicant: Fridababy, LLC, Miami, FL (US)

(72) Inventors: Sophia FORNARO, Miami, FL (US); John FRITZ, Buffalo, NY (US); Bailey DENTON, Miami, FL (US); Chelsea HIRSCHHORN, Miami Beach, FL (US)

(21) Appl. No.: 19/025,712

(22) Filed: Jan. 16, 2025

Related U.S. Application Data
(60) Provisional application No. 63/702,016, filed on Oct. 1, 2024.

(51) Int. Cl. A61M 1/00

(52) U.S. Cl. CPC A61M 1/67; A61M 2210/0618

(57) ABSTRACT

A nasal suction device includes a collection chamber within a nasal tip for collecting mucus. The nasal suction device also includes an air pump, located within a main body, in fluid communication with the collection chamber. The nasal suction device further includes an air gap chamber defined in a base removably coupled with the main body, the secondary air gap chamber positioned between the collection chamber and the air pump.

### CROSS-REFERENCE TO RELATED APPLICATION

The present application claims the benefit of U.S. Provisional Patent Application No. 63/702,016, filed on Oct. 1, 2024, and titled “ELECTRIC NASAL SUCTION DEVICE WITH SECONDARY AIR GAP CHAMBER,” the disclosure of which is expressly incorporated by reference in its entirety.

### BACKGROUND

### Field

Aspects of the present disclosure generally relate to nasal suction devices, and more specifically to nasal suction devices that incorporate a secondary air gap chamber for protecting an internal air pump from liquid intrusion.

### Background

In most cases, conventional nasal suction devices include a single collection chamber for trapping mucus and other debris removed from a nasal passage. This collection chamber serves as a primary defense against liquid entering the device's internal mechanisms, such as an air pump or power source. In some cases, mucus or liquid may bypass the collection chamber and enter the device's internal mechanisms. When liquid infiltrates the device's internal mechanisms, the liquid may interfere with the pump's operation, leading to decreased performance, clogging, or even premature pump failure due to moisture damage.

### SUMMARY

A nasal suction device includes a collection chamber within a suction nozzle for collecting mucus. The nasal suction device also includes an air pump, located within a main body, in fluid communication with the collection chamber. The nasal suction device further includes an air gap chamber defined in a base removably coupled with the main body, the secondary air gap chamber positioned between the collection chamber and the air pump.

In some aspects a method for collecting mucus from a nose includes placing a tip of a suction nozzle to the nose, such that mucus is extracted from the nose via suction generated by an air pump housed in a main body. The main body may be removeable attached to a base including an air gap chamber defined within an air gap chamber housing, a hose connector extending from the main body. The suction nozzle may be attached to a first end of a hose. A second end of the hose may be attached to the hose connector. The method also includes detaching the base from the main body to access the air gap chamber. The method further includes cleaning mucus collected within the air gap chamber.

This has outlined, rather broadly, the features and technical advantages of the present disclosure in order that the detailed description that follows may be better understood. Additional features and advantages of the present disclosure will be described below. It should be appreciated by those skilled in the art that this present disclosure may be readily utilized as a basis for modifying or designing other structures for carrying out the same purposes of the present disclosure. It should also be realized by those skilled in the art that such equivalent constructions do not depart from the teachings of the present disclosure as set forth in the appended claims. The novel features, which are believed to be characteristic of the present disclosure, both as to its organization and method of operation, together with further objects and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

The features, nature, and advantages of the present disclosure will become more apparent from the detailed description set forth below when taken in conjunction with the drawings in which like reference characters identify correspondingly throughout.

FIG. 1 is a diagram illustrating an example of a nasal suction device, in accordance with various aspects of the present disclosure.

FIG. 2 is a diagram illustrating an example of a disassembly process for the base of a nasal suction device, in accordance with various aspects of the present disclosure

FIG. 3A illustrates an example of a bottom shell of the air pump housing, in accordance with various aspects of the present disclosure.

FIG. 3B illustrates a detailed view of the connection point, in accordance with various aspects of the present disclosure.

FIG. 3C illustrates an example of the bottom shell of the air pump housing, in accordance with various aspects of the present disclosure.

FIG. 4A illustrates a detailed top view of the detachable base of the nasal suction device, in accordance with various aspects of the present disclosure.

FIG. 4B illustrates a detailed view of the detachable base, in accordance with various aspects of the present disclosure.

FIG. 4C provides a detailed perspective view of the detachable base, in accordance with various aspects of the present disclosure.

FIG. 5 provides an internal view of the bottom shell of the nasal suction device, in accordance with various aspects of the present disclosure.

FIGS. 6A, 6B, and 6C are diagrams illustrating examples of a nozzle docked with an air pump housing, in accordance with various aspects of the present disclosure.

FIG. 7 is a diagram illustrating examples of a geometry of a distal end of a soft-tip suction nozzle, in accordance with various aspects of the present disclosure.

FIG. 8 is a flow diagram illustrating an example of a process for using a nasal suction device, in accordance with various aspects of the present disclosure

### DETAILED DESCRIPTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. It will be apparent to those skilled in the art, however, that these concepts may be practiced without these specific details. In some instances, well-known structures and components are shown in block diagram form in order to avoid obscuring such concepts.

Based on the teachings, one skilled in the art should appreciate that the scope of the present disclosure is intended to cover any aspect of the present disclosure, whether implemented independently of or combined with any other aspect of the present disclosure. For example, an apparatus may be implemented, or a method may be practiced using any number of the aspects set forth. In addition, the scope of the present disclosure is intended to cover such an apparatus or method practiced using other structure, functionality, or structure and functionality in addition to, or other than the various aspects of the present disclosure set forth. It should be understood that any aspect of the present disclosure may be embodied by one or more elements of a claim.

The word “exemplary” is used herein to mean “serving as an example, instance, or illustration.” Any aspect described herein as “exemplary” is not necessarily to be construed as preferred or advantageous over other aspects.

Although particular aspects are described herein, many variations and permutations of these aspects fall within the scope of the present disclosure. Although some benefits and advantages of the preferred aspects are mentioned, the scope of the present disclosure is not intended to be limited to particular benefits, uses, or objectives. Rather, aspects of the present disclosure are intended to be broadly applicable to different technologies, system configurations, networks, and protocols, some of which are illustrated by way of example in the figures and in the following description of the preferred aspects. The detailed description and drawings are merely illustrative of the present disclosure rather than limiting, the scope of the present disclosure being defined by the appended claims and equivalents thereof.

In most cases, conventional nasal suction devices include a single collection chamber for trapping mucus and other debris removed from a nasal passage. This collection chamber serves as a primary defense against liquid entering the device's internal mechanisms, such as an air pump or power source. In some cases, mucus or liquid may bypass the collection chamber and enter the device's internal mechanisms. When liquid infiltrates the device's internal mechanisms, the liquid may interfere with the pump's operation, leading to decreased performance, clogging, or even premature pump failure due to moisture damage. To mitigate this issues, some conventional nasal suction devices may use liquid-tolerant air pumps. These liquid-tolerant air pumps are often more complex and expensive to manufacture. Additionally, liquid-tolerant air pumps may fail when exposed to excessive liquid, making liquid-tolerant air pumps less reliable in the long term. As a result of the aforementioned design limitations, conventional nasal suction devices often require frequent replacement or professional servicing to maintain functionality.

It may be desirable to improve a nasal suction device to protect the air pump from liquid ingress while also providing user-friendly access for regular cleaning and maintenance, thereby enhancing both the durability and hygiene of the device. Various aspects of the present disclosure are directed to a nasal suction device that includes both a primary collection chamber and a secondary collection chamber, which may also be referred to as an air gap chamber (hereinafter used interchangeably). The air gap chamber is designed to be user-accessible, allowing for easy cleaning and maintenance. This air gap chamber acts as a buffer between the primary collection chamber and the air pump, effectively preventing any liquid, such as mucus, from reaching the pump. This protective barrier helps extend the overall life of the nasal suction device by safeguarding the internal components.

In some examples, a base of the air pump unit may be removably coupled to the air gap chamber. In some such examples, the base may feature a quarter-turn removal mechanism that enables convenient access to the air gap chamber, allowing users to disassemble the device quickly for cleaning. This design promotes easy maintenance, making it suitable for regular use while ensuring that the air pump remains protected from potential liquid damage. The quarter-turn mechanism simplifies the cleaning process, enhancing the usability and longevity of the device by reducing the likelihood of liquid reaching critical components. Aspects of the present disclosure are not limited to a quarter-turn removal mechanism. Other mechanisms may be used to remove the base from the air pump unit.

Particular aspects of the subject matter described in this disclosure can be implemented to realize one or more of the following potential advantages. In some examples, the described techniques, such as the incorporation of a user-accessible air gap chamber and a quarter-turn removal mechanism, may enhance the case of maintenance, reduce the risk of pump failure due to liquid ingress, and improve overall device hygiene. Furthermore, by providing a clear separation between the collection chamber and the air pump, these techniques ensure that the device remains reliable and functional over extended periods, reducing the need for frequent replacements or costly repairs. This design is especially beneficial in maintaining the sanitary condition of a nasal suction device, which is critical when used for infants and young children.

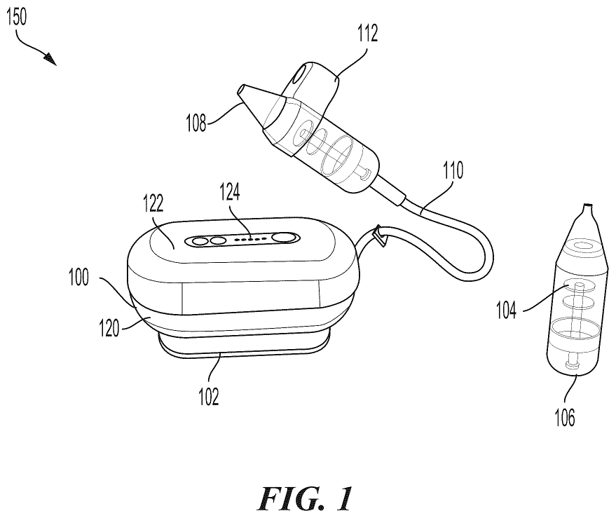

FIG. 1 is a diagram illustrating an example of a nasal suction device 150, in accordance with various aspects of the present disclosure. The nasal suction device 150 (also referred to as an electric nose suction device or an electric nasal suction device, hereinafter used interchangeably) includes an air pump housing unit 100, a removable base 102, a suction nozzle 108, and a hose 110 connecting the suction nozzle 108 to the air pump housing 100 via a connection on the removable base 102. The air pump housing unit 100 may also be referred to as the air pump housing (herein after used interchangeably). The suction nozzle 108 may be interchangeable with other nozzles, such as a high reach soft tip suction nozzle 106. The air pump housing unit 100 has a generally rectangular shape with rounded edges and corners.

The suction nozzle 108 is ergonomically designed to comfortably fit into an infant's or young child's nostrils. In some examples, the high-reach soft-tip suction nozzle 106 may be used instead of the suction nozzle 108. The high-reach soft-tip suction nozzle 106 extends deeper into the nostril for improved mucus clearance. This soft tip is constructed of flexible silicone or similar material to ensure a gentle and safe user experience, reducing the risk of irritation or discomfort during use. The high-reach soft-tip suction nozzle is particularly beneficial in cases of severe congestion, as it allows for the effective removal of mucus from deeper nasal passages.

Both types of suction nozzles 106 and 108 further incorporate a primary umbrella-style diverter 104, positioned near the tip of the nozzle. This diverter 104 acts as a first line of defense, preventing mucus and other liquids from progressing further into the device's airflow system. By intercepting liquid at the nozzle, the diverter significantly reduces the risk of contamination or damage to downstream components, such as an air pump and power supply housed within the air pump housing unit 100.

In some examples, a finger loop 112 may encircle one of the suction nozzles 106 or 108. The finger loop 112 may be constructed from a soft, flexible material such as silicone. The finger loop 112 is an example of a no-slip grip finger loop that provides a secure and stable hold, even when dealing with a squirming or uncooperative infant. The finger loop 112 may be ergonomically designed to accommodate various hand sizes.

The nasal suction device 150 also includes a hose 110 that connects one of the suction nozzles 106 or 108 to the air pump housing unit 100, providing a continuous and efficient airflow pathway. The hose 110 may include a premium connection to the attached suction nozzle 106 or 108, constructed from thermoplastic polyurethane (TPU), silicone, or another flexible elastomer material, which facilitates quick and easy detachment from the suction nozzle 106 or 108.

The air pump housing unit 100 houses the air pump (not shown in the example of FIG. 1), which serves as the primary driver of suction. The air pump is securely enclosed within the air pump housing unit 100, which may be compact and portable for ease of use. The removable base 102 may be positioned at the base of the air pump housing unit 100. The removable base 102 provides access to a secondary air gap chamber (not shown in the example of FIG. 1). The base 102 may be removed via a twist-off mechanism, allowing for easy disassembly and cleaning. The inclusion of the secondary air gap chamber acts as an additional safeguard, trapping any liquid that bypasses the primary diverter, and preventing it from reaching the air pump.

The removable base 102 enables users to access the air gap chamber for cleaning and maintenance. This feature improves hygiene and reduces the risk of bacterial buildup, which is desirable when using the device 150 on infants and young children. The twist-off mechanism is designed for quick and intuitive operation, such that users can easily maintain the device without the need for specialized tools or expertise.

The air pump housing unit 100 is composed of a bottom shell 120 and an upper shell 122, which together enclose and protect the internal components of the nasal suction device, such as, for example, an air pump, exhaust hose, air pump hose, power source, and/or other electronics. The bottom shell 120 provides the foundation for mounting internal components, such as the air pump 312, air intake hose 500, exhaust port hose 316, and other associated features. The upper shell 122, on the other hand, completes the housing structure and includes user-facing features for controlling the operation of the device.

The upper shell 122 includes an interface 124 to facilitate user interaction and control. This interface 124 may include an on/off switch for powering the device, a suction power control switch to adjust the suction strength as needed, and additional features such as an LCD screen, status indicators, or other functional elements. The LCD screen may provide information such as battery life, current suction settings, and operational status. Status indicators, such as LED lights, may inform the user about the device's charging state, readiness for use, or alerts related to maintenance, such as when the air gap chamber 400 needs cleaning.

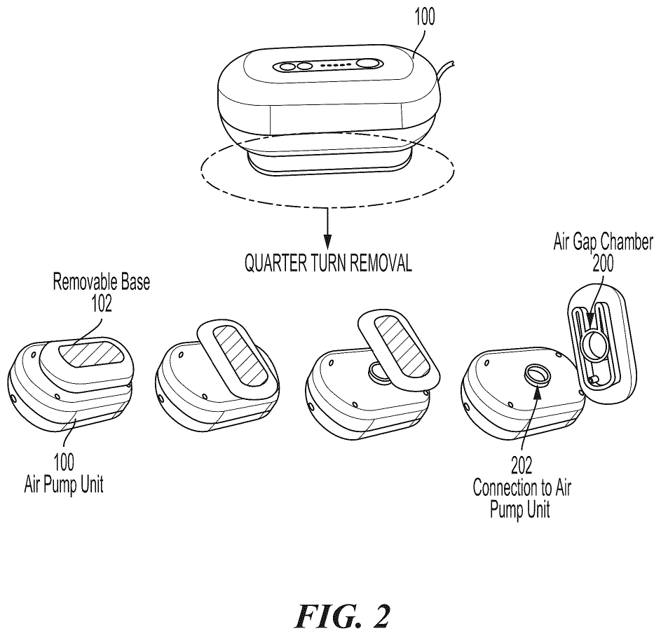

FIG. 2 is a diagram illustrating an example of a disassembly process for the base 102 of a nasal suction device 150, in accordance with various aspects of the present disclosure. As discussed, nasal suction device 150 includes an air pump housing unit 100 with a removable base 102, which houses the secondary air gap chamber 200. The air pump housing unit 100 encloses critical internal components, including the air pump and associated electronics, such as the power source. The base 102 is detachable to facilitate access to the air gap chamber 200. The base 102 has a generally oval shape. Still, other shapes are contemplated. In the example of FIG. 2, the air gap chamber 200 is shown without a cover.

In some examples, a rotational motion may securely attach or detach the base 102 from the air pump housing unit 100. As the base 102 rotates, it gradually disengages from a connection point 202 on the air pump housing unit 100. This rotational engagement may use a threaded connection, such as a male and female thread interface, where the base 102 includes an internal thread (female component) that mates with an external thread (male component) on the connection point 202 of the air pump housing unit 100. The threaded connection may provide a secure and tight fit during operation, preventing accidental detachment while allowing smooth and reliable disassembly when necessary. In other examples, the connection may incorporate a bayonet-style locking mechanism, where the base 102 is equipped with tabs or lugs that slide into corresponding grooves on the connection point 202. A partial rotational motion aligns the tabs with the grooves to engage the base securely, and a reverse motion unlocks the connection for easy removal. Alternatively, the base 102 may use a snap-fit mechanism with flexible retaining clips or hooks that engage a corresponding ridge or groove on the connection point 202. Aspects of the present disclosure are not limited to the aforementioned engagement mechanisms. Other types of engagement mechanisms are contemplated. Regardless of the specific engagement mechanism, each example provides a reliable and secure (e.g., airtight) connection between the base 102 and the air pump housing unit 100 during operation while allowing for efficient and user-friendly disassembly to access the air gap chamber.

FIG. 2 also illustrates the progressive detachment of the base 102 (e.g., removeable base 102), specifically illustrating how the base 102 transitions from being fully attached to the air pump housing unit 100 to completely removed. Once rotated via the quarter-turn, the base 102 detaches from the air pump housing unit 100, exposing the secondary air gap chamber 200 located on an underside of the base 102. The underside is a side of the base 102 that faces the air pump housing unit 100 when the base 102 is attached to the air pump unit. The secondary air gap chamber 200 is defined in an enclosure or recess of the base 102. As discussed, the secondary air gap chamber 200 serves as a barrier, intercepting any liquid, such as mucus, that bypasses the primary collection chamber.

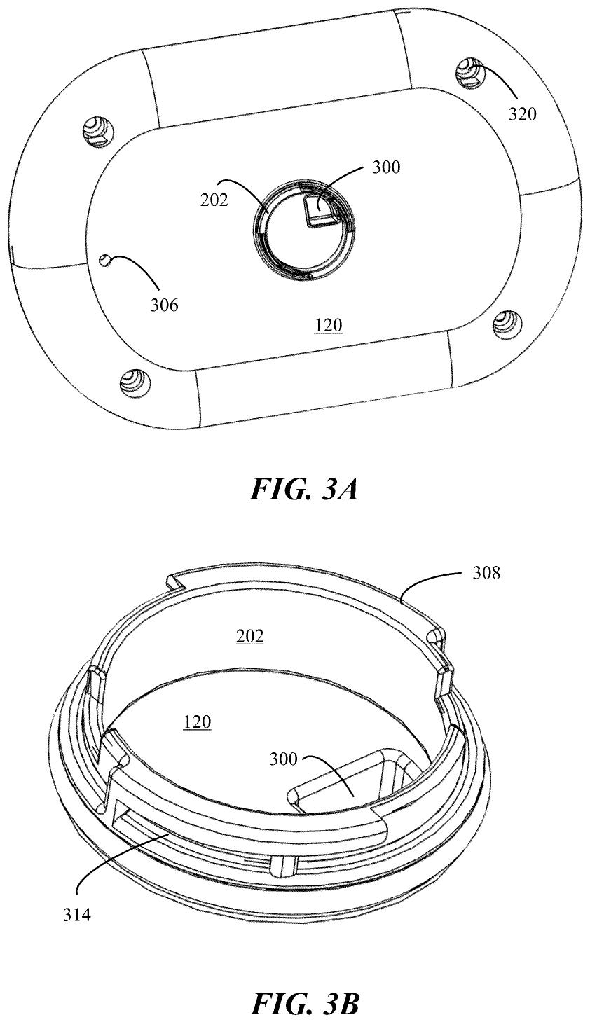

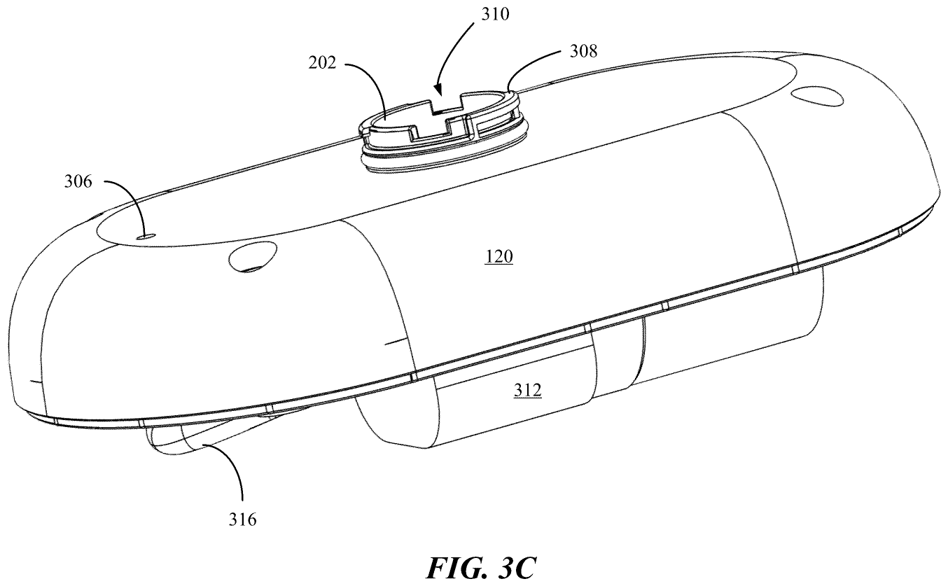

FIG. 3A illustrates an example of a bottom shell 120 of the air pump housing 100, in accordance with various aspects of the present disclosure. The bottom shell 120 works in conjunction with a top shell (not shown in the example of FIG. 3A) to protect internal mechanisms, such as an air pump, power supply, and other components. The bottom shell 120 also provides a functional interface for connecting the detachable base. As shown in the example of FIG. 3A, at a center of the bottom shell 120 is an air inlet 300, which serves as an entry point for air drawn through the suction nozzle and the secondary air gap chamber into the air pump. Surrounding the air inlet 300 is the connection point 202, which protrudes outward from the surface of the bottom shell 120 to facilitate secure attachment to the detachable base (not shown in the example of FIG. 3A). The connection point 202 includes grooves, ridges, and/or threads that align with corresponding elements on the detachable base, ensuring a secure connection (e.g., airtight connection) during operation while allowing for smooth engagement and disengagement via a quarter-turn mechanism. The bottom shell 120 also includes an exhaust port 306, which serves as the exit point for air expelled from the air pump. The exhaust port 306 is positioned to expel air safely away from the device and the user. Additionally, the bottom shell 120 features multiple attachment points 320 distributed around its perimeter, which may include screw holes for attaching to the top shell.

FIG. 3B illustrates a detailed view of the connection point 202, in accordance with various aspects of the present disclosure. The connection point 202 is used to secure the detachable base to the bottom shell of the air pump housing. The connection point 202 is designed to align and couple with the air gap chamber (not shown in FIG. 3B) while ensuring a secure and airtight interface during operation.

As shown in FIG. 3B, the connection point 202 includes two ridges 308 that extend outwardly from its surface. Each ridge 308 may engage with a corresponding female connector on the air gap chamber. Additionally, each ridge 308 only extends along a portion of a circumference of the connection point 202. The interaction between the ridge 308 and the female connector provides a locking mechanism that secures the connection point 202 to the air gap chamber, ensuring stability and preventing unintentional disconnection during operation. The connection point 202 may include additional structural features, such as notches 310 and/or grooves 314, to enhance its locking capability with the air gap chamber.

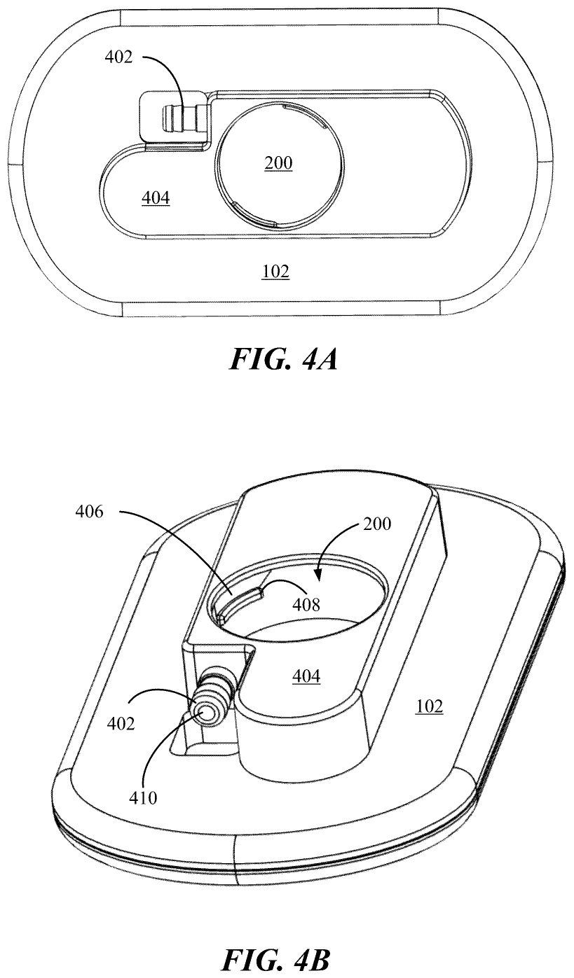

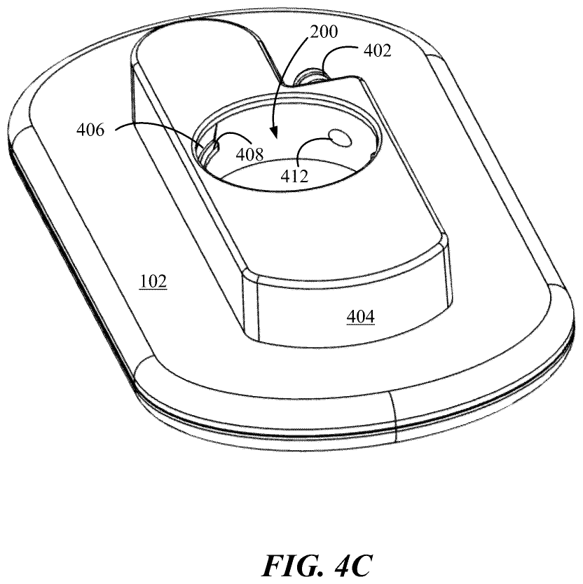

FIG. 3C illustrates an example of the bottom shell 120 of the air pump housing, in accordance with various aspects of the present disclosure. As shown in the example of FIG. 3C, the connection point 202 extends outward (e.g., perpendicularly) from the surface of the bottom shell 120. The connection point 202 may securely attach the base unit via corresponding connection points in the air gap chamber. For example, each ridge 308 may interact with a corresponding female connector within the air gap chamber base, creating a secure locking mechanism to ensure stability during operation. Additionally, the connection point 202 includes notches 310. The notches 310 align with an air inlet, such as an air inlet 412 described with reference to FIG. 4C, when in an assembled configuration to allow air/fluid passage into the secondary chamber.

As shown in the example of FIG. 3C, the bottom shell 120 houses the air pump 312, which drives the suction process. Specifically, the air pump 312 generates negative pressure, drawing air through the air inlet and expelling it through the exhaust port via the exhaust port hose 316. The air pump 312 may be supported by vibration-dampening mounts to ensure quiet operation and minimize mechanical stress on surrounding components. The exhaust port hose 316 connects the air pump 312 to the exhaust port, which is positioned on the surface of the bottom shell 120. The exhaust port hose 316 may be constructed from flexible, durable material to facilitate smooth air transfer while preventing leaks or blockages.

FIG. 4A illustrates a detailed top view of the detachable base 102 of the nasal suction device, in accordance with various aspects of the present disclosure. As shown in the example of FIG. 4A, the base 102 includes the air gap chamber 400, an air gap chamber housing 404, and a hose connector 402. The air gap chamber 400 is centrally positioned within the base 102 and serves as a secondary containment system to trap any liquid, such as mucus, that bypasses the primary collection chamber. The air gap chamber 400 is positioned to align with the connection point of the bottom shell.

The design of the air inlet, such as the air inlet 300 described with reference to FIGS. 3A and 3B, relative to the air gap chamber 400 improves an ability of the nasal suction device to prevent mucus or other liquid from entering the air pump. The small opening of the air inlet, positioned centrally within the air gap chamber 400, serves as a deliberate bottleneck in the airflow pathway. This feature leverages the size discrepancy between the relatively larger volume of the air gap chamber 400 and the restricted diameter of the air inlet to effectively reduce the likelihood of liquid infiltration. When the device operates, mucus and other liquids entering the secondary air gap chamber 400 are subject to gravitational forces and surface tension, which cause the liquid to settle at the bottom of the chamber rather than being drawn toward the air inlet 300. The chamber's larger internal volume allows it to act as a collection reservoir, providing sufficient space for liquids to pool while maintaining unobstructed airflow through the air inlet. This design minimizes the velocity and upward motion of liquid particles, making it inherently difficult for mucus to reach the air inlet. Additionally, the small size of the air inlet creates a high-pressure differential as air flows into the pump. This pressure differential is optimized for airflow but is insufficient to overcome the surface tension of liquid pooling in the chamber, effectively preventing mucus or liquid droplets from being sucked through the narrow opening. The size and placement of the air inlet also contribute to the chamber's ability to separate liquids from the airflow, such that clean, dry air passes through to the pump.

As shown in the example of FIG. 4A, the air gap chamber housing 404 surrounds the air gap chamber 400. The air gap chamber housing 404 extends outward from the base 102 in a generally perpendicular orientation. The housing 404 encloses the air gap chamber 400, providing structural protection and ensuring proper alignment with the air inlet. The outward extension of the housing 404 from the base 102 creates a secure interface that isolates the air gap chamber from other components while allowing for easy cleaning and maintenance. The housing 404 may be constructed from a durable material to withstand frequent use and repeated disassembly.

Additionally, a hose connector 402, which may be ridged, is positioned adjacent to the air gap chamber 400 and is designed to connect securely to the hose that attaches to the suction nozzle. The ridged design of the hose connector 402 provides enhanced grip and scaling, preventing air leaks and ensuring a stable connection during operation. The connector 402 may include multiple ridges along its length to accommodate varying hose diameters and to provide a secure fit that resists accidental disconnection.

FIG. 4B illustrates a detailed view of the detachable base 102, in accordance with various aspects of the present disclosure. As previously discussed, the air gap chamber 400 is centrally positioned and enclosed by the air gap chamber housing 404, which extends outward from the base 102. The housing 404 provides structural protection and ensures proper alignment of the air gap chamber 400 with the air inlet defined within the connector, such as the connection point 202 described with reference to FIGS. 2, 3A, 3B, and 3C.

As shown in the example of FIG. 4B, an upper region of the air gap chamber 400 includes a female connector 406, which is specifically designed to receive a ridge of the connector. Two female connectors 406 may be defined on an interior wall of the air gap chamber 400. This female connector 406 ensures that the air gap chamber 400 remains securely and accurately positioned when the base 102 is attached to the bottom shell 120. Additionally, the air gap chamber 400, may include one or more ridges 408 that extend outward from a wall of the air gap chamber 400. This ridge 408 is designed to engage with a corresponding groove on the connection point, such as a groove 314 described with reference to FIG. 3B. The interaction between the ridge 408 and the groove provides an additional layer of stability, preventing misalignment and ensuring a tight, secure fit. The ridge 408 may be defined below the female connector 406.

Additionally, as shown in the example of FIG. 4B, the hose connector 402 is located adjacent to the air gap chamber 400 and extends outward from the air gap chamber housing 404. The hose connector 402 has an opening 410 at a distal end. Another opening may be defined on a wall of the air gap chamber 400, such that a hollow space extends from the opening 410 at the distal end of the hose connector 402 to the opening defined on the wall of the air gap chamber 400.

FIG. 4C provides a detailed perspective view of the detachable base 102, in accordance with various aspects of the present disclosure. As previously discussed, The air gap chamber 400 is centrally located within the base 102 and surrounded by the air gap chamber housing 404, which extends outward to provide structural integrity and proper alignment with the main unit. The air gap chamber 400 includes one or more female connector 406, designed to securely engage a corresponding ridge of the connection point (not shown), ensuring a stable and airtight connection. Additionally, inside the air gap chamber 400, one or more ridges 408 extend outward to engage with a corresponding groove on the connection point, adding further stability and preventing misalignment.

The hose connector 402, positioned adjacent to the air gap chamber 400, features a ridged surface for securely attaching the hose leading to the suction nozzle. An interior opening 412, defined on the wall of the air gap chamber 400, creates a pathway for airflow between the distal end of the hose connector 402 and the interior of the air gap chamber 400. This hollow space facilitates the transfer of air drawn through the suction nozzle and hose into the air gap chamber 400. The positioning of the interior opening 412 ensures that air flows efficiently from the hose connector 402 into the air gap chamber 400.

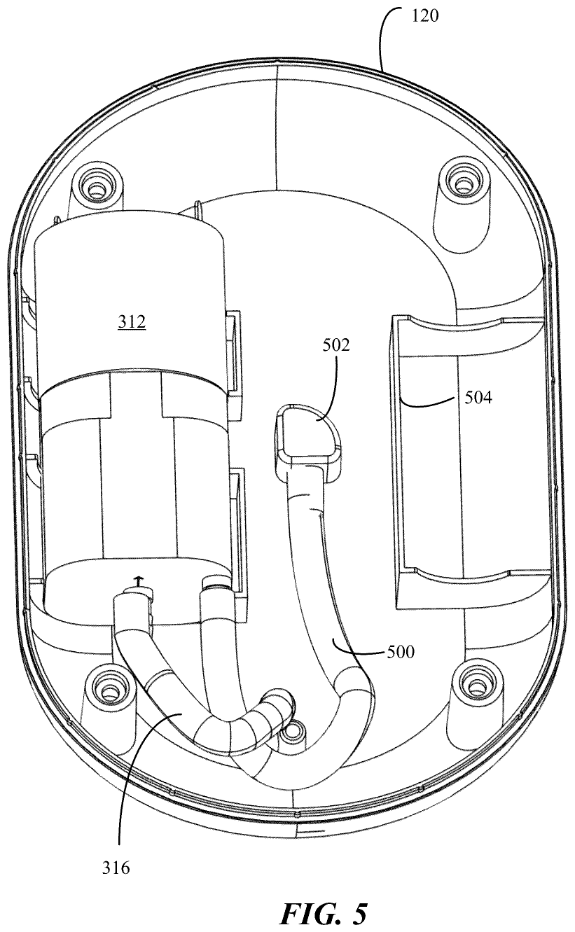

FIG. 5 provides an internal view of the bottom shell 120 of the nasal suction device, in accordance with various aspects of the present disclosure. As shown in the example of FIG. 5, the air pump 312 is mounted within the bottom shell 120 and serves as the primary component for generating suction. Air drawn from the suction nozzle and the air gap chamber enters the air pump 312 through the air intake hose 500, which is connected at one end to the air inlet cover 502 and at the other end to the intake port of the air pump 312. The air inlet cover 502, positioned near the center of the main unit, provides a secure interface for connecting the air intake hose 500, ensuring an airtight seal that maintains optimal suction performance. The air intake hose 500 may be constructed from flexible, durable material to prevent air leakage while efficiently channeling airflow out of the device. The air inlet cover 502 covers an opening created by the air inlet defined on an outer surface of the bottom shell 120. As noted, air enters the air inlet cover 502 via a channel formed from the air inlet. As shown in FIG. 5, the exhaust port hose 316, extending from the outlet of the air pump 312, may direct expelled air to the exhaust port on the main unit's housing. The exhaust port hose 316 may be constructed from flexible, durable material to prevent air leakage while efficiently channeling airflow out of the device. A housing 504 may be defined on a side opposing the air pump 312. The housing 504 may provide a secure compartment for the power source or other electronic components required to operate the device. The housing 504 may also include insulation or vibration-dampening features to minimize interference from the air pump during operation.

As discussed, the nasal suction device features a secondary air gap chamber, such as the air gap chamber 200 described with reference to FIGS. 2, 4A, 4B, and 4C. The air gap chamber is designed to capture any liquid, such as mucus, that bypasses the primary umbrella-style diverter located within the suction nozzle. Positioned centrally within the detachable base and enclosed by the air gap chamber housing, the air gap chamber acts as a safeguard to prevent liquid from reaching the air inlet. By creating this protective barrier, the air gap chamber ensures that liquid does not enter and damage the air pump, thereby preserving the functionality and durability of the device. Unlike conventional designs, which often rely on sealed or difficult-to-access components, the air gap chamber 400 in this device is user-accessible, as shown in FIGS. 4A and 4B. The nasal suction device also incorporates a quarter-turn removal system, which enables the removable base to be easily detached for cleaning and maintenance. As detailed in FIGS. 4A and 4C, the base includes additional features like the hose connector, which securely attaches to the hose connected to the suction nozzle.

The nasal suction device is designed with a versatile power source to ensure reliable and convenient operation. The device may be powered through a direct electrical connection (plug-in) or via internal rechargeable or replaceable batteries.

In some configurations, the device includes a power cord that connects to a standard electrical outlet. This configuration ensures a consistent and uninterrupted power supply to the air pump and other internal components, such as control circuitry. In some examples, a power port may be located on the external housing, allowing for the power cord to be easily connected and disconnected. An internal power management system may ensure that the supplied current is appropriately regulated, preventing overvoltage or damage to the internal components.

In other configurations, the device is equipped with an internal battery compartment that houses one or more rechargeable or replaceable batteries. These batteries may be lithium-ion, nickel-metal hydride (NiMH), or alkaline, depending on the specific implementation. The battery compartment is accessible via a panel on the underside or back of the main unit, allowing users to easily replace batteries if necessary.

For rechargeable batteries, the device includes a charging port, which may use a standard charging interface, such as USB-C, micro-USB, or a proprietary adapter. The internal battery management system monitors charge levels and prevents overcharging, enhancing the battery's lifespan. A battery indicator, such as an LED or display panel on the top of the main unit, informs the user of the remaining charge level, ensuring the device is ready for use when needed.

Some implementations may offer a dual power option, allowing the device to switch seamlessly between plug-in and battery operation. This feature enables the user to operate the device in a fixed location with a power outlet or on the go without access to electricity. When connected to a power outlet, the device may simultaneously charge its internal battery while in use, ensuring continuous operation.

To ensure safety, the device may be equipped with protective features, such as short-circuit protection, overload protection, and thermal management systems. These features safeguard the device from electrical faults and overheating during extended operation. Additionally, energy-efficient circuitry may be used to minimize power consumption, making the device environmentally friendly and cost-effective.

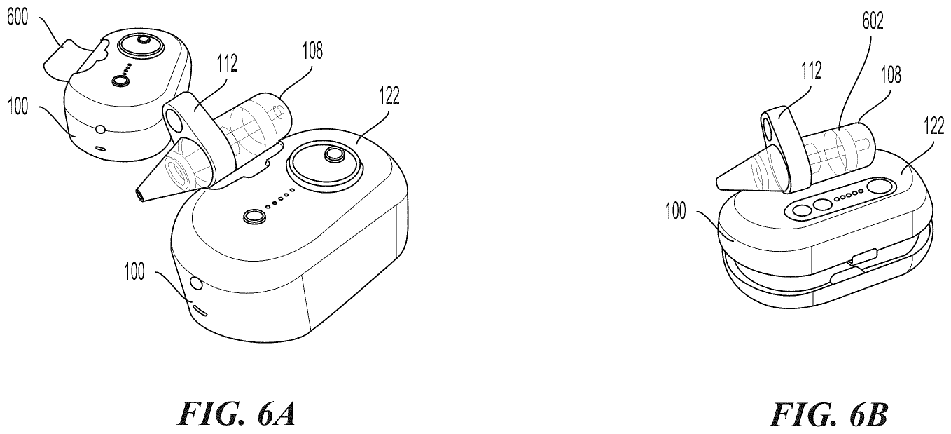

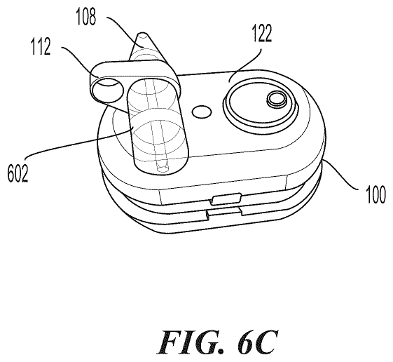

In some examples, a nozzle, such as a suction nozzle 108 or a soft-tip suction nozzle 106 as described with reference to FIG. 1, may be docked (e.g., attached) to an air pump housing unit. FIG. 6A is a diagram illustrating an example of a nozzle docked with to an air pump housing unit, in accordance with various aspects of the present disclosure. As shown in the example of FIG. 6A, the air pump housing unit 100 may include a hinged dock 600 that is pivotally attached to an upper shell 122 of the air pump housing unit 100. The suction nozzle 108 may be housed in the hinged dock 600. Of course, other types of nozzles, such as the soft-tip suction nozzle 106 described with reference to FIG. 1, may be housed in the hinged dock 600.

In some other examples, a suction nozzle may be inlaid on the air pump housing unit 100. FIGS. 6B and 6C are diagrams illustrating examples of a nozzle docked with to an air pump housing unit, in accordance with various aspects of the present disclosure. As shown in the examples of FIGS. 6B and 6C, a suction nozzle 108 may be docked (e.g., attached) to an upper shell 122 of the air pump housing unit 100. In some such examples, the upper shell 122 may include a concave area for receiving the suction nozzle 108. Additionally, or alternatively, the suction nozzle 108 may be docked via a magnetic attachment mechanism, or another mechanism. For example, the suction nozzle 108 may include a metal ring 602, so that the nozzle 108 may be attracted to a magnet included in the upper shell 122. Of course, other types of nozzles, such as the soft-tip suction nozzle 106 described with reference to FIG. 1, may be docked with the upper shell 122.

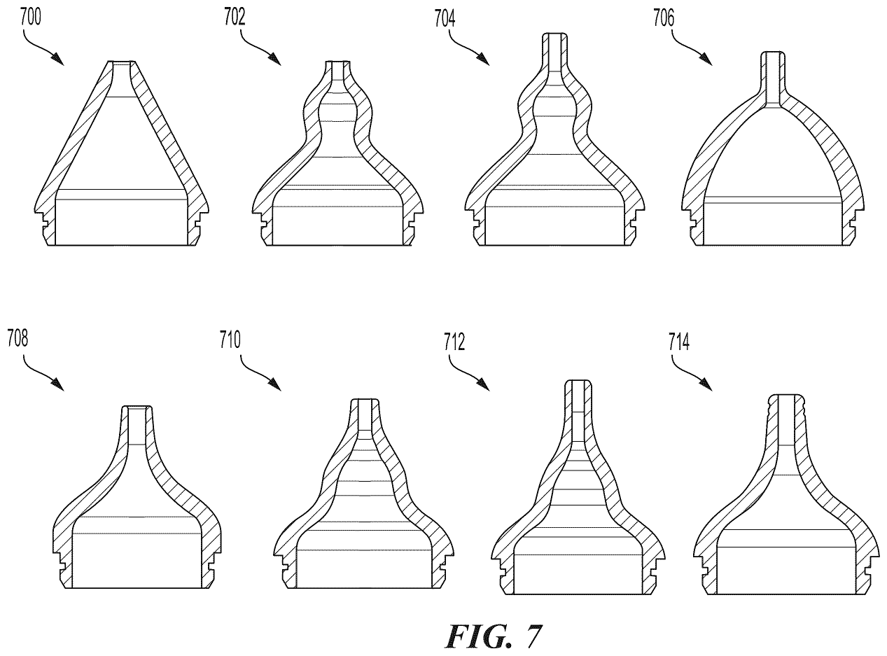

As shown in the example of FIG. 1, a distal end of a soft-tip suction nozzle 106 may be cone shaped. Aspects of the present disclosure are not limited to a cone shaped distal end. Other shapes are contemplated. FIG. 7 is a diagram illustrating examples of a geometry of a distal end of a soft-tip suction nozzle, in accordance with various aspects of the present disclosure. As shown in the example of FIG. 7, a distal end of a soft-tip suction nozzle may have a cone shape 700. Still, other shapes 702, 704, 706, 708, 710, 712, and 714 may be used for a distal end of the soft-tip suction nozzle, such as the soft-tip suction nozzle 106 described with reference to FIG. 1.

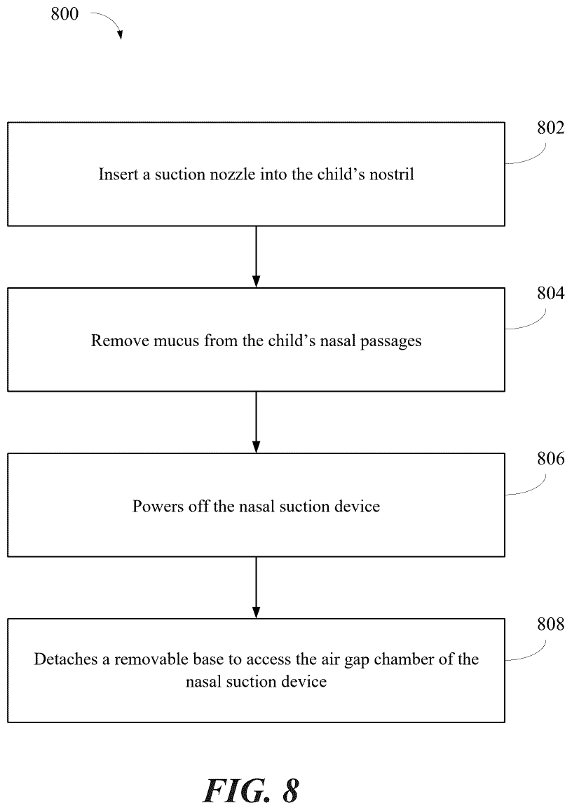

FIG. 8 is a flow diagram illustrating an example of a process 800 for using a nasal suction device, in accordance with various aspects of the present disclosure. The nasal suction device may be an example of the nasal suction device described with reference to FIG. 1. The process begins at block 802 by inserting a suction nozzle (e.g., nasal tip) into the child's nostril. Aspects of the present disclosure are not limited to inserting the suction nozzle into a child's nostril, the suction nozzle may also be used on adults. The nozzle may include a soft silicone tip, ensures a comfortable and non-invasive experience for the child. The user may grip the nozzle using a finger loop, which provides stability and prevents accidental slipping during operation. Prior to, or after inserting the suction nozzle, the user powers on the device via an interface of the air pump housing unit. This interface may include an on/off switch, suction power controls, and/or status indicators. When activated, the air pump generates suction, creating a negative pressure system. Airflow is directed from the nozzle through the hose and into the secondary air gap chamber, passing through the air inlet into the air pump. Expelled air exits through the exhaust port hose.

At block 804, the suction process begins as the device removes mucus from the child's nasal passages. The umbrella-style diverter, integrated into the suction nozzle, acts as the first barrier, blocking large debris from entering the airflow pathway. Any liquid or smaller particles bypassing the diverter are directed into the secondary air gap chamber, where they are trapped. The small opening of the air inlet, such as the air inlet 300, as described with reference to FIGS. 3A and 3B, prevents liquid from advancing into the pump, safeguarding the internal mechanisms.

In some examples, the user may monitor the hose connected to the suction nozzle to observe the flow of mucus and determine if mucus is bypassing the umbrella-style diverter. The transparency of the hose provides an early warning system, allowing the user to pause operation and empty the air gap chamber before overfilling occurs. If necessary, the suction strength can be adjusted using the controls on the interface. This feature allows the user to tailor the suction to the child's comfort level or the severity of the nasal congestion, ensuring optimal mucus removal.

At block 806, after completing the suction process, the user powers off the device using the interface. This action halts the airflow and allows for safe disassembly of the device. At block 808, using, for example, the quarter-turn removal mechanism, the user detaches the removable base to access the air gap chamber. In some examples, the suction nozzle, hose, and air gap chamber are cleaned using warm soapy water to remove any mucus or debris. All components are dried completely to prevent bacterial growth or contamination during storage.

As discussed, various aspects of the present disclosure are directed to a nasal suction device for safely and effectively removing mucus from a nose, particularly for use with infants and young children. The device comprises a suction nozzle with a collection chamber to collect mucus, which is fluidly connected to an air pump housed within a main body. A secondary air gap chamber, defined within a removable base coupled to the main body, is positioned between the collection chamber and the air pump to serve as a safeguard against liquid ingress. The air gap chamber traps liquid that bypasses a primary diverter, such as an umbrella-style diverter, located within the collection chamber of the suction nozzle. This design protects the air pump from potential damage caused by liquid and ensures the device maintains functionality.

In some examples, the base housing the air gap chamber is detachable via a quarter-turn mechanism, allowing users to easily access and clean the air gap chamber after use. A transparent hose, detachably coupled between the suction nozzle and the base, allows users to monitor the movement of liquid and serves as an early warning system to prevent overfilling. The suction nozzle may include a tapered or flexible distal end to ensure comfort and fit during use and may be dockable with the main body for storage. The air pump can be initiated through a user interface located on the main body, allowing the user to control suction power and operation conveniently. The associated method for using the device includes placing the suction nozzle at the nose to extract mucus via suction generated by the air pump, which directs airflow through the hose and air gap chamber before reaching the pump. After use, the base can be detached, and the air gap chamber can be cleaned to remove collected mucus, ensuring hygienic reuse.

As used herein, the term “determining” encompasses a wide variety of actions. For example, “determining” may include calculating, computing, processing, deriving, investigating, looking up (e.g., looking up in a table, a database or another data structure), ascertaining and the like. Additionally, “determining” may include receiving (e.g., receiving information), accessing (e.g., accessing data in a memory) and the like. Furthermore, “determining” may include resolving, selecting, choosing, establishing, and the like.

As used herein, a phrase referring to “at least one of” a list of items refers to any combination of those items, including single members. As an example, “at least one of: a, b, or c” is intended to cover: a, b, c, a-b, a-c, b-c, and a-b-c.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

It is to be understood that the claims are not limited to the precise configuration and components illustrated above. Various modifications, changes, and variations may be made in the arrangement, operation, and details of the methods and apparatus described above without departing from the scope of the claims.

What is claimed is:

1. A nasal suction device comprising:
a collection chamber within a suction nozzle for collecting mucus;
an air pump, located within a main body, in fluid communication with the collection chamber; and
an air gap chamber defined in a base removably coupled with the main body, the secondary air gap chamber positioned between the collection chamber and the air pump.

2. The nasal suction device of claim 1, wherein the air gap chamber is accessible to a user for cleaning.

3. The nasal suction device of claim 1, wherein the base is detachable via a quarter-turn.

4. The nasal suction device of claim 1, further comprising a transparent hose having one end detachable coupled to the base and one end detachable coupled with the suction nozzle.

5. The nasal suction device of claim 1, wherein the air gap chamber is configured to trap liquid bypassing a primary diverter positioned in the collection chamber.

6. The nasal suction device of claim 5, wherein the primary diverter is an umbrella-style diverter.

7. The nasal suction device of claim 1, wherein the air gap chamber is positioned over an air inlet coupled to the air pump via an air inlet hose.

8. The nasal suction device of claim 1, wherein a distal end of the suction nozzle is flexible.

9. The nasal suction device of claim 1, wherein a distal end of the suction nozzle is tapered.

10. The nasal suction device of claim 1, wherein the main body includes a dock for docking the suction nozzle.

11. A method for collecting mucus from a nose, comprising:
placing a tip of a suction nozzle to the nose, such that mucus is extracted from the nose via suction generated by an air pump housed in a main body, the main body being removeable attached to a base including an air gap chamber defined within an air gap chamber housing, a hose connector extending from the main body, the suction nozzle attached to a first end of a hose, a second end of the hose attached to the hose connector;
detaching the base from the main body to access the air gap chamber; and
cleaning mucus collected within the air gap chamber.

12. The method of claim 11, wherein the base is detachable via a quarter-turn.

13. The method of claim 11, wherein:
the hose is transparent; and
the hose is detachable connected to the suction nozzle and the hose connector.

14. The method of claim 11, wherein the air gap chamber traps liquid bypassing a primary diverter positioned in a collection chamber of the suction nozzle.

15. The method of claim 14, wherein the primary diverter is an umbrella-style diverter.

16. The method of claim 11, wherein the air gap chamber is positioned over an air inlet coupled to the air pump via an air inlet hose.

17. The method of claim 11, further comprising initiating the air pump prior to or after placing the tip of the suction nozzle to the nose.

18. The method of claim 17, wherein the air pump is initiated via user input received at the main body.

19. The method of claim 11, wherein a distal end of the nasal tip is flexible.

20. The method of claim 11, wherein the main body includes a dock for docking the nasal tip.
